(19) United States
(12) Patent Application Publication
(10) Pub. No.: US 2001/0049449 A1
(43) Pub. Date: Dec. 6, 2001

(54) Aromatic sulfonyl alpha-cycloamino hydroxamates and their use as MMP inhibitors

(76) Inventors: Daniel P. Becker, Glenview, IL (US); Madeleine H. Li, Vernon Hills, IL (US); Gary A. DeCrescenzo, St. Charles, MO (US)

Correspondence Address: Pharmacia Corporation, Attention: David M. Gryte, Corporate Patent Department, P.O. Box 5110, Chicago, IL, 60680, US

(21) Appl. No.: 09/778,411

(22) Filed: Feb. 7, 2001

Related U.S. Application Data
(63) Continuation-in-part of application No. 09/254,530, filed on Dec. 23, 1999, now abandoned.

(51) Int. Cl. C 07D 2 7/12

(52) U.S. Cl. 548/537

(57) ABSTRACT

This invention is directed to aromatic sulfonyl α-cycloamino hydroxamates (also known as aromatic sulfonyl α-cycloamino hydroxamic acids) that, inter alia, inhibit matrix metalloproteinase (also known as matrix metalloprotease or MMP) activity. This invention also is directed to a prevention or treatment method that comprises administering an aromatic sulfonyl α-cycloamino hydroxamate in an MMP-inhibiting effective amount to a mammal having (or disposed to having) a pathological condition associated with MMP activity.

### PRIORITY CLAIM TO RELATED PATENT APPLICATION

This patent claims priority from U.S. patent application Ser. No. 09/254,530 (filed on Mar. 4, 1998). The entire text of that patent application is incorporated by reference into this patent.

### FIELD OF THE INVENTION

This invention is directed generally to proteinase (also known as protease) inhibitors, and, more particularly, to aromatic sulfonyl α-cycloamino hydroxamates (also known as aromatic sulfonyl α-cycloamino hydroxamic acids) that, inter alia, inhibit matrix metalloproteinase (also known as matrix metalloprotease or MMP) activity. This invention also is directed to compositions of such inhibitors, intermediates for the syntheses of such inhibitors, methods for making such inhibitors, and methods for preventing or treating conditions associated with pathological MMP activity.

### BACKGROUND OF THE INVENTION

Connective tissue is a required component of all mammals. It provides rigidity, differentiation, attachments, and, in some cases, elasticity. Connective tissue components include, for example, collagen, elastin, proteoglycans, fibronectin, and laminin. These biochemicals make up (or are components of) structures, such as skin, bone, teeth, tendon, cartilage, basement membrane, blood vessels, cornea, and vitreous humor.

Under normal conditions, connective tissue turnover and/or repair processes are in equilibrium with connective tissue production. Degradation of connective tissue is carried out by the action of proteinases released from resident tissue cells and/or invading inflammatory or tumor cells.

MMPs, a family of zinc-dependent proteinases, make up a major class of enzymes involved in degrading connective tissue. MMPs are divided into classes, with some members having several different names in common use. Examples are: MMP-1 (also known as collagenase I, fibroblast collagenase, or EC 3.4.24.3); MMP-2 (also known as gelatinase A, 72 kDa gelatinase, basement membrane collagenase, or EC 3.4.24.24), MMP-3 (also known as stromelysin 1 or EC 3.4.24.17), proteoglycanase, MMP-7 (also known as matrilysin), MMP-8 (also known as collagenase II, neutrophil collagenase, or EC 3.4.24.34), MMP-9 (also known as gelatinase B, 92 kDa gelatinase, or EC 3.4.24.35), MMP-10 (also known as stromelysin 2 or EC 3.4.24.22), MMP-11 (also known as stromelysin 3), MMP-12 (also known as metalloelastase, human macrophage elastase, or HME), MMP-13 (also known as collagenase III), and MMP-14 (also known as membrane MMP).

Excessive breakdown of connective tissue by MMPs is a feature of many pathological conditions. Inhibition of MMPs provides a control mechanism for tissue decomposition to prevent and/or treat these pathological conditions. Such pathological conditions include, for example, rheumatoid arthritis, osteoarthritis, septic arthritis, multiple sclerosis, corneal ulceration, epidermal ulceration, gastric ulceration, tumor metastasis, tumor invasion, angiogenesis, periodontal disease, proteinuria, Alzheimer's disease, coronary thrombosis, bone disease, and defective injury repair (e.g., weak repairs, adhesions such as post-surgical adhesions, and scarring).

MMPs also are involved in the biosynthesis of tumor necrosis factor (TNF). TNFs are implicated in many pathological conditions. TNF-α, for example, is a cytokine that is presently thought to be produced initially as a 28 kD cell-associated molecule. It is released as an active, 17 kD form that can mediate a large number of deleterious effects in vitro and in vivo. TNF-α can cause and/or contribute to the effects of inflammation, rheumatoid arthritis, autoimmune disease, multiple sclerosis, graft rejection, fibrotic disease, cancer, infectious diseases, malaria, mycobacterial infection, meningitis, fever, psoriasis, cardiovascular/pulmonary effects (e.g., post-ischemic reperfusion injury), congestive heart failure, hemorrhage, coagulation, hyperoxic alveolar injury, radiation damage, and acute phase responses like those seen with infections and sepsis and during shock (e.g., septic shock and hemodynamic shock). Chronic release of active TNF-α can cause cachexia and anorexia. TNF-α also can be lethal.

Inhibiting TNF (and related compounds) production and action is an important clinical disease treatment. MMP inhibition is one mechanism that can be used. MMP (e.g., collagenase, stromelysin, and gelatinase) inhibitors, for example, have been reported to inhibit TNF-α release. See Gearing et al. Nature 376, 555-557 (1994); and McGeehan et al., Nature 376, 558-561 (1994). MMP inhibitors also have been reported to inhibit TNF-α convertase, a metalloproteinase involved in forming active TNF-α. See WIPO Int'l Pub. Nos. WO 94/24140, WO 94/02466, and WO 97/20824.

MMPs also are involved in other biochemical processes in mammals. These include control of ovulation, postpartum uterine involution, possibly implantation, cleavage of APP (β-amyloid precursor protein) to the amyloid plaque, and inactivation of α₁-protease inhibitor (α₁-PI). Inhibiting MMPs permits control of fertility. In addition, increasing and maintaining the levels of an endogenous or administered serine protease inhibitor (e.g., (α₁-PI) supports the treatment and prevention of pathological conditions such as emphysema, pulmonary diseases, inflammatory diseases, and diseases of aging (e.g., loss of skin or organ stretch and resiliency).

Numerous metalloproteinase inhibitors are known.

Metalloproteinase inhibitors include, for example, natural biochemicals, such as tissue inhibitor of metalloproteinase (TIMP), α₂-macroglobulin, and their analogs and derivatives. These are high-molecular-weight protein molecules that form inactive complexes with metalloproteinases.

A number of smaller peptide-like compounds also have been reported to inhibit metalloproteinases. Mercaptoamide peptidyl derivatives, for example, have been reported to inhibit angiotensin converting enzyme (also known as ACE) in vitro and in vivo. ACE aids in the production of angiotensin II, a potent pressor substance in mammals. Inhibiting ACE leads to lowering of blood pressure.

Thiol-containing amide or peptidyl amide-based MMP inhibitors also have been reported. See, e.g., WO95/12389; WO96/11209; and U.S. Pat. No. 4,595,700. Additionally, hydroxamate-containing MMP inhibitors have been reported. See, e.g., WO 95/29892, WO 97/24117, WO 97/49679, and EP 0 780 386 (disclosing carbon back-boned compounds). See also, WIPO Int'l Pub. Nos. WO 90/05719, WO 93/20047, WO 95/09841, WO 96/06074, Schwartz et al., Progr. Med. Chem., 29:271-334(1992); Rasmussen et al., Pharmacol. Ther., 75(1): 69-75 (1997); and Denis et al., Invest. New Drugs, 15(3): 175-185 (1997) (disclosing hydroxamates that have peptidyl back-bones or peptidomimetic back-bones).

It is often advantageous for an MMP inhibitor drug to target certain MMPs over others. For example, it is typically preferred to inhibit MMP-2, MMP-3, MMP-9, and/or MMP-13 when treating and/or preventing of cancer, inhibiting of metastasis, and inhibiting angiogenesis. It also is typically preferred to inhibit MMP-13 when preventing and/or treating osteoarthritis. See, e.g., Mitchell et al., J. Clin. Invest., 97:761-768 (1996); and Reboul et al., J. Clin. Invest., 97:2011-2019 (1996). Normally, however, it is preferred to use a drug that has little or no inhibitory effect on MMP-1. This preference stems from the fact that MMP-1 is involved in several homeostatic processes, and inhibition of MMP-1 consequently tends to interfere with such processes.

Many known MMP inhibitors exhibit the same or similar inhibitory effects against each of the MMPs. For example, batimastat (a peptidomimetic hydroxamate) has been reported to exhibit IC₅₀ values of from about 1 to about 20 nM against each of MMP-1, MMP-2, MMP-3, MMP-7, and MMP-9. Marimastat (another peptidomimetic hydroxamate) has been reported to be another broad-spectrum MMP inhibitor with an enzyme inhibitory spectrum similar to batimastat, except that Marimastat exhibited an IC₅₀ value against MMP-3 of 230 nM. See Rasmussen et al., Pharmacol. Ther., 75(l): 69-75 (1997).

Meta analysis of data from Phase I/II studies using Marimastat in patients with advanced, rapidly progressive, treatment-refractory solid tumor cancers (colorectal, pancreatic, ovarian, and prostate) indicated a dose-related reduction in the rise of cancer-specific antigens used as surrogate markers for biological activity. Although Marimastat exhibited some measure of efficacy via these markers, toxic side effects reportedly were observed. The most common drug-related toxicity of Marimastat in those clinical trials was musculoskeletal pain and stiffness, often commencing in the small joints in the hands, and then spreading to the arms and shoulder. A short dosing holiday of 1-3 weeks followed by dosage reduction reportedly permits treatment to continue. See Rasmussen et al., Pharmacol. Ther., 75(1): 69-75 (1997). It is thought that the lack of specificity of inhibitory effect among the MMPs may be the cause of that effect.

In view of the importance of hydroxamate MMP inhibitors in the treatment of several pathological conditions and the lack of enzyme specificity exhibited by 2 of the more potent drugs that have been in clinical trials, it would be a great benefit if hydroxamates of greater enzyme specificity could be found. This would particularly be true if the hydroxamates exhibited strong inhibitory activity against MMP-2, MMP-9 and/or MMP-13 (i.e., MMPs associated with several pathological conditions), while exhibiting little or no inhibition of MMP-1. The following disclosure describes hydroxamate MMP inhibitors that exhibit such desirable activities.

### SUMMARY OF THE INVENTION

This invention is directed to compounds that inhibit MMP activity, particularly compounds that inhibit MMP-2, MMP-9. and/or MMP-13, while generally exhibiting relatively little or no inhibition against MMP-1 activity. This invention also is directed to a method for treating a mammal (e.g., a mouse; rat; rabbit; cat; dog; horse; or primate such as a monkey, chimpanzee, or human) having a condition associated with pathological MMP activity.

Briefly, therefore, one embodiment of this invention is directed to an aromatic sulfonyl α-cycloamino hydroxamate or salt thereof that can act as an MMP inhibitor. In a preferred embodiment, the hydroxamate has a structure selected from the group consisting of:

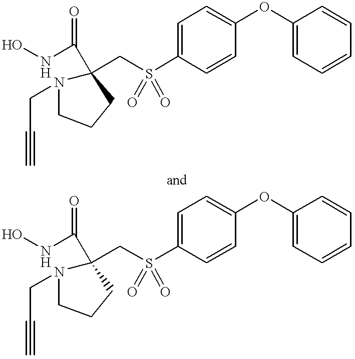

Another embodiment of this invention is directed to pharmaceutical compositions comprising a hydroxamate shown above or a pharmaceutically acceptable salt thereof.

A further embodiment of this invention is directed to a method for preventing or treating a pathological condition associated with pathological MMP activity in a mammal. This method comprises administering a hydroxamate shown above or a pharmaceutically acceptable salt thereof in an effective amount to the mammal.

A particularly preferred embodiment of this invention is directed to a method for inhibiting MMP-2 activity selectively over MMP-1 activity in a mammal by administering a hydroxamate shown above or a pharmaceutically acceptable salt thereof. Another particularly preferred embodiment is directed to a method for inhibiting MMP-13 activity selectively over MMP-1 activity in a mammal by administering a hydroxamate shown above or a pharmaceutically acceptable salt thereof. In an even more particularly preferred embodiment, the invention is directed to a method for inhibiting both MMP-2 and MMP-13 activity selectively over MMP-1 activity in a mammal by administering a hydroxamate shown above or a pharmaceutically acceptable salt thereof.

An additional embodiment of this invention is directed to a method for preparing a medicament, which comprises a hydroxamate shown above or a pharmaceutically acceptable salt thereof, for preventing and/or treating a condition in a mammal associated with pathological MMP activity. A particularly preferred embodiment is directed to a method for preparing a medicament, which comprises a hydroxamate shown above or a pharmaceutically acceptable salt thereof, for preventing or treating a condition where there is an advantage in inhibiting MMP-2 and/or MMP-13 activity selectively over MMP-1 activity.

This invention is beneficial, in part, because it provides for compounds, compositions, and treatments that are effective in inhibiting MMP activity, particularly MMP activity implicated in pathological conditions involving excessive breakdown of connective tissue. Even more particularly, this invention is beneficial because it provides for compounds, compositions, and treatments that are effective for inhibiting MMP-13 and/or MMP-2. Inhibition of such MMPs is beneficial because they are associated with pathological conditions such as, for example, rheumatoid arthritis, osteoarthritis, septic arthritis, corneal ulceration, epidermal ulceration, gastric ulceration, tumor metastasis, tumor invasion, tumor angiogenesis, periodontal disease, proteinuria, multiple sclerosis, Alzheimer's disease, coronary thrombosis, and bone disease. This invention is also particularly beneficial because it provides for compounds, compositions, and treatments that are effective for treating such pathological conditions by selective inhibition of an MMP(s) (e.g., MMP-13 and/or MMP-2) associated with such conditions, while causing minimal side effects from inhibiting other proteinases (e.g., MMP-1), whose activity is necessary or desirable for normal body function.

Further benefits of this invention will be apparent to one skilled in the art from reading this disclosure.

### DESCRIPTION OF THE PREFERRED EMBODIMENTS

MMP Inhibitors

In accordance with this invention, it has been found that certain aromatic sulfonyl α-cycloamino hydroxamates are effective for inhibiting MMPs associated with excessive (or otherwise pathological) breakdown of connective tissue. In particular, it has been found that these hydroxamates tend to be effective for inhibiting MMP-2 and MMP-13, which can be particularly destructive to tissue if present or generated in abnormally excessive quantities or concentrations.

Moreover, it has been discovered that these hydroxamates tend to be selective toward inhibiting MMP-2 and/or MMP-13 (as well as other MMPs associated with pathological condition conditions), and avoid excessive inhibition of other MMPs (particularly MMP-1) essential to normal bodily function (e.g., tissue turnover and repair). This is illustrated in Example 3 below.

One embodiment of this invention is directed to an aromatic sulfonyl α-cycloamino hydroxamate generally corresponding in structure to the following Formula (IV):

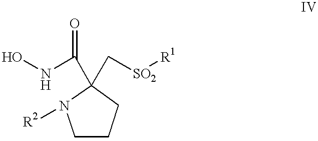

In Formula IV, R² preferably is hydrogen, C₁-C₈ hydrocarbyl, C₁-C₆ hydrocarbyloxycarbonyl C₁-C₄ hydrocarbyl, aryl C₁-C₄ hydrocarbyl, heteroaryl C₁-C₄ hydrocarbyl, aryloxy C₁-C₄ hydrocarbyl, or heteroaryloxy C₁-C₄ hydrocarbyl. More preferably, R² is hydrogen, C₃-C₆ cyclohydrocarbyl, t-butoxycarbonyl, phenethyl, 2-propynyl, or 3-methoxybenzyl.

Also in Formula IV, R¹ preferably comprises a 5- or 6-membered cyclohydrocarbyl, heterocyclo, aryl, or heteroaryl group bonded directly to the depicted SO₂-group. Exemplary 5- or 6-membered cyclohydrocarbyl, heterocyclo, aryl, or heteroaryl groups that can constitute a portion of R¹ include phenyl; 2-, 3-, or 4-pyridyl; 2-naththyl; 2-pyrazinyl; 2- or 5-pyrimidinyl; 2- or 3-benzo(b)thienyl; 8-purinyl; 2- or 3-furyl; 2- or 3-pyrrolyl; 2-imidazolyl; cyclopentyl; cyclohexyl; 2- or 3-piperidinyl; 2- or 3-morpholinyl; 2- or 3-tetrahydropyranyl; 2-imidazolidinyl; 2- or 3-pyrazolidinyl; and the like. A phenyl group is particularly preferred and is therefore used illustratively herein.

R¹ also preferably has a length (i.e., the distance from the depicted SO₂-group to the point on R¹ furthest from the depicted SO₂-group along the longest chain of atoms in R¹) that is greater than about that of a fully-extended saturated 6-carbon chain (i.e., a hexyl group) and less than about that of a fully-extended saturated 20-carbon chain (i.e., an eicosyl group). More preferably, R¹ has a length that is equivalent to the length of a fully-extended saturated chain of from about 8 to about 18 carbon atoms. The length of R¹ is believed to play a role in the overall activity and selectivity of a contemplated inhibitor. A compound having an R¹ that is shorter in length than an octyl group (e.g., a 4-methoxyphenyl group) typically exhibits moderate to poor inhibitory activity against all the MMP, whereas a compound having an R¹ that is at least as long as an octyl chain (e.g. a 4-phenoxyphenyl group that has a length of about a nine-carbon chain) tends to exhibit good to excellent potencies against MMP-13 and/or MMP-2, and be selective toward MMP-13 and/or MMP-2 over MMP-1.

The length of R¹ can be readily determined using published bond angles, bond lengths, and atomic radii; or by building models using commercially available kits whose bond angles, lengths, and atomic radii are in accord with accepted, published values. It should be recognized that a single-ring or fused-ring cyclohydrocarbyl, heterocyclo, aryl, or heteroaryl group is not itself long enough to fulfill the preferred length requirement for R¹. Thus, such a cyclohydrocarbyl, heterocyclo, aryl, or heteroaryl group is preferably itself substituted.

In addition to the foregoing preferred length, R¹ also preferably has a structure such that if it were rotated about an axis drawn through the SO₂-bonded 1-position and the 4-position of a 6-membered ring group or drawn through the SO₂-bonded 1-position and the center of 3,4-bond of a 5-membered ring group, it would define a volume having a widest dimension in a direction transverse to the axis of rotation that is from about that of one furanyl ring to about that of 2 phenyl rings. When utilizing this preferred width criterion, a fused ring system such as a naphthyl or purinyl group is considered to be a 6- or 5-membered ring that is substituted at appropriate positions numbered from the SO₂-linkage that is deemed to be at the 1-position. Thus, a 2-naphthyl substituent or an 8-purinyl substituent falls within the preferred rotational width criterion. On the other hand, a 1-naphthyl group or a 7- or 9-purinyl group is too large upon rotation to fall within the preferred rotational width criterion.

Groups falling within the length and width preferences of R¹ include, for example, 4-(phenyl)phenyl [biphenyl], 4-(4′-methoxyphenyl)phenyl, 4-(phenoxy)phenyl, 4-(thiophenyl)phenyl [4-(phenylthio)phenyl], 4-(phenylazo)phenyl 4-(phenylureido)phenyl, 4-(anilino)phenyl, 4-(nicotinamido)phenyl, 4-(isonicotinamido)phenyl, 4-(picolinamido)phenyl, and 4-(benzamido)phenyl, with 4-(phenoxy)phenyl and 4-(thiophenyl)phenyl being more preferred.

Two particularly preferred compounds are:

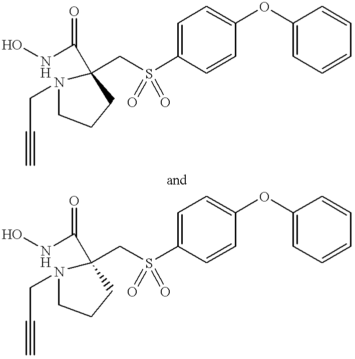

These two enantiomers may be separated using a chiral high performance liquid chromatography column. For example, the nonohydrochloride salts of the enantiomers can be introduced into such a column to form trifluoroacetate salts which exit the column at different times. The separation may, for example, be conducted under the following conditions:

(a) a mixture of monohydrochloride salts of both compounds is introduced with an isocratic eluent into the column;

(b) 34.8 to 35% (by weight) of the eluent consists of ethanol, 64.8 to 65% (by weight) of the eluent consists of heptane, and 0.2% (by weight) of the eluent consists of trifluoroacetic acid;

(c) the column has a 250 mm length and a 22.5 mm inside diameter; and

(d) the column is packed with silica gel particles, which have a 10 micron particle size and are coated with amylose tris(3,5-dimethylphenyl carbamate).

Such a separation is demonstrated in Example 2 below. The trifluoroacetate salt exiting the column first under such conditions is typically the salt of the more preferred enantiomer (i.e., the first-exiting enantiomer). This first-exiting enantiomer, in fact, is typically preferred over both the second-exiting enantiomer (i.e., the enantiomer whose trifluoroacetate salt exits second) and a racemic mixture of the enantiomers. This preference stems from the surprising discovery that the first-exiting enantiomer has greater potency toward MMP-2 and MMP-13 than the other enantiomer or a racemic mixture of the two enantiomers. It also stems from the fact that the first-exiting enantiomer has a greater selectivity toward MMP-2 and MMP-13 than the other enantiomer or a racemic mixture of the two enantiomers. See Example 3.

It should be noted, however, that in some embodiments, the second-exiting enantiomer may be more preferred, given that it tends to exhibit little, if any, potency toward MMP-1 compared to the first-exiting enantiomer or the racemic mixture. See Example 3.

MMP Inhibitor Salts

The compounds of this invention can be used in the form of salts derived from inorganic or organic acids. These salts include, for example, the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxy-ethanesulfonate, lactate, maleate, methanesulfonate, nicotinate, 2-naphthalenesulfonate, oxalate, palmoate, pectinate, persulfate, 3-phenylpropionate, picrate, pivalate, propionate, succinate, tartrate, thiocyanate, tosylate, mesylate, and undecanoate. Also, the basic nitrogen-containing groups can be quaternized with agents such as lower alkyl (C₁-C₆) halides (e.g., methyl, ethyl, propyl, and butyl chlorides, bromides, and iodides), dialkyl sulfates (e.g., dimethyl, diethyl, dibutyl, and diamyl sulfates), long chain halides (e.g., decyl, lauryl, myristyl, and stearyl chlorides, bromides, and iodides), aralkyl halides (e.g., benzyl and phenethyl bromides), and others. Water or oil-soluble or dispersible products are thereby obtained as desired. The salts are formed by combining the basic compounds with the desired acid.

Other compounds of this invention that are acids also can form salts. Examples include salts with alkali metals or alkaline earth metals (e.g., sodium, potassium, calcium, or magnesium) or with organic bases or basic quaternary ammonium salt. In some cases, the salts also can be used as an aid in the isolation, purification, and/or resolution of the compounds.

Salts of this invention include, for example, trifluoroacetate salts such as:

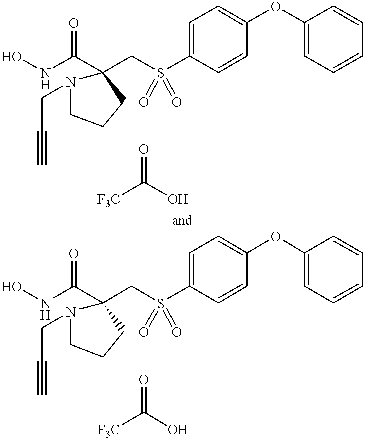

Salts of this invention also include, for example, monohydrochloride salts such as:

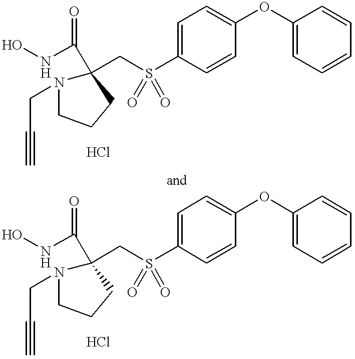

Preventing or Treating Pathological Conditions Using MMP Inhibitors

One embodiment of this invention is directed to a process for preventing or treating a pathological condition associated with MMP activity in a mammal (e.g., a human or in a veterinary context such as a farm, companion, or wild animal). This process comprises administering a hydroxamate or pharmaceutically acceptable salt thereof described above in an amount effective to inhibit a target MMP(s) in a mammal disposed to having or having such a condition. In many instances, this administration will be repeated a plurality of times. Here, “preventing a condition” means reducing the risk of (or delaying) the onset of the condition in a mammal that does not have the condition, but is predisposed to having the condition. In contrast, “treating a condition” means ameliorating, suppressing, or eradicating an existing condition. The pathological condition may be (a) the result of pathological MMP activity itself, and/or (b) affected by MMP activity (e.g., diseases associated with TNF-α).

The preferred total daily dose of the hydroxamate or salt thereof administered to a mammal (in single or divided doses) is typically from about 0.001 to about 100 mg/kg, more preferably from about 0.001 to about 30 mg/kg, and even more preferably from about 0.01 to about 10 mg/kg (i.e., mg hydroxamate or salt thereof per kg body weight). Dosage unit compositions can contain such amounts or submultiples thereof to make up the daily dose. Multiple doses per day typically may be used to increase the total daily dose, if desired.

Factors affecting the preferred dosage regimen include the type, age, weight, sex, diet, and condition of the patient; the severity of the pathological condition; the route of administration; pharmacological considerations, such as the activity, efficacy, pharmacokinetic, and toxicology profiles of the particular hydroxamate or salt thereof employed; whether a drug delivery system is utilized; and whether the hydroxamate or salt thereof is administered as part of a drug combination. Thus, the dosage regimen actually employed can vary widely, and, therefore, can deviate from the preferred dosage regimen set forth above.

A hydroxamate or salt thereof useful in this invention can be formulated as a pharmaceutical composition. Such a composition can, for example, be administered orally, parenterally, by inhalation spray, rectally, or topically as a formulation containing conventional pharmaceutically acceptable carriers, adjuvants, and vehicles, as desired. Formulation of drugs is generally discussed in, for example, Hoover, John E., Remington's Pharmaceutical Sciences, Mack Publishing Co. (Easton, Pa.: 1975); and Liberman, H. A. and Lachman, L., eds., Pharmaceutical Dosage Forms, Marcel Decker (New York, N.Y.: 1980).

Solid dosage forms for oral administration include, for example, capsules, tablets, pills, powders, and granules. In such solid dosage forms, the hydroxamates or salts thereof are ordinarily combined with one or more adjuvants. If administered per os, the hydroxamates or salts thereof can be mixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation, as can be provided in a dispersion of the hydroxamate or salt thereof in hydroxypropylmethyl cellulose. In the case of capsules, tablets, and pills, the dosage forms also can comprise buffering agents, such as sodium citrate, or magnesium or calcium carbonate or bicarbonate. Tablets and pills additionally can be prepared with enteric coatings.

Liquid dosage forms for oral administration include, for example, pharmaceutically acceptable emulsions, solutions, suspensions, syrups, and elixirs containing inert diluents commonly used in the art (e.g. water). Such compositions also can comprise adjuvants, such as wetting, emulsifying, suspending, flavoring (e.g., sweetening), and/or perfuming agents.

“Parenteral administration” includes subcutaneous injections, intravenous injections, intramuscular injections, intrasternal injections, and infusion. Injectable preparations (e.g., sterile injectable aqueous or oleaginous suspensions) can be formulated according to the known art using suitable dispersing, wetting agents, and/or suspending agents. Acceptable vehicles and solvents include, for example, water, 1,3-butanediol, Ringer's solution, isotonic sodium chloride solution, bland fixed oils (e.g., synthetic mono- or diglycerides), fatty acids (e.g., oleic acid), dimethyl acetamide, surfactants (e.g., ionic and non-ionic detergents), and/or polyethylene glycols.

Formulations for parenteral administration may, for example, be prepared from sterile powders or granules having one or more of the carriers or diluents mentioned for use in the formulations for oral administration. The hydroxamates or salts thereof can be dissolved in water, polyethylene glycol, propylene glycol, ethanol, corn oil, cottonseed oil, peanut oil, sesame oil, benzyl alcohol, sodium chloride, and/or various buffers.

Suppositories for rectal administration can be prepared by, for example, mixing the drug with a suitable nonirritating excipient such as cocoa butter; synthetic mono- di- or triglycerides; fatty acids; and/or polyethylene glycols that are solid at ordinary temperatures, but liquid at the rectal temperature and will therefore melt in the rectum to release the drug.

“Topical administration” includes the use of transdermal administration, such as transdermal patches or iontophoresis devices.

Other adjuvants and modes of administration well-known in the pharmaceutical art may also be used.

Definitions

“Hydrocarbyl” means a straight-chain aliphatic group; branched-chain aliphatic group; alicyclic group; or a straight-chain aliphatic group, branched-chain aliphatic group, alicyclic group substituted with one or more straight-chain aliphatic groups, branched-chain aliphatic groups, and/or alicyclic groups. Thus, alkyl, alkenyl, and alkynyl groups are contemplated, whereas aromatic hydrocarbons such as phenyl and naphthyl groups are referred to herein as aryl groups. Examples of alkyl groups include methyl, ethyl, n-propyl, isopropyl, n-butyl, isobutyl, sec-butyl, tert-butyl, pentyl, iso-amyl, hexyl, octyl, and the like. Examples of suitable alkenyl groups include ethenyl (vinyl), 2-propenyl, 3-propenyl, 1,4-pentadienyl, 1,4-butadienyl, 1-butenyl, 2-butenyl, 3-butenyl, decenyl, and the like. Examples of alkynyl groups include ethynyl, 2-propynyl, 3-propynyl, decynyl, 1-butynyl, 2-butynyl, 3-butynyl, and the like.

Usual chemical suffix nomenclature is followed when using the word “hydrocarbyl” except that the usual practice of removing the terminal “yl” and adding an appropriate suffix is not always followed because of the possible similarity of a resulting name to one or more substituents. Thus, a hydrocarbyl ether is referred to as a “hydrocarbyloxy” group rather than a “hydrocarboxy” group as may possibly be more proper when following the usual rules of chemical nomenclature. On the other hand, a hydrocarbyl group containing a —C(O)O— functionality is referred to as a hydrocarboyl group inasmuch as there is no ambiguity in using that suffix. A substituent that cannot exist (e.g., a C₁ alkenyl group) is not intended to be encompassed by “hydrocarbyl”.

“Carbonyl” (alone or in combination) means —C(═O)—, wherein the 2 terminal bonds (valences) are independently substituted.

“Thiol” or “sulfhydryl” (alone or in combination) means —SH.

“Thio” or “thia” (alone or in combination) means a thiaether (i.e., an ether group wherein the ether oxygen atom is replaced by a sulfur atom).

“Amino” (alone or in combination) means —NH₂. “Mono-substituted amino” (alone or in combination) means a substituted amine wherein one hydrogen atom is replaced with a substituent (i.e., —N(H)(substituent)), and “di-substituted amine” means an amine wherein 2 hydrogen atoms of an amino group are replaced with independently selected substituent groups (—N(substituent)₂).

“Quaternary amine” means a nitrogen with 4 substituents that is positively charged and accompanied by a counter ion (i.e. —N⁺(substituent)₄).

“Hydroxyl” (alone or in combination) means —OH.

“Nitro” (alone or in combination) means —NO₂.

“Azo” (alone or in combination) means —N═N—, wherein the bonds at the remaining 2 bonds are independently substituted.

“Sulfonyl” (alone or in combination) means —S(═O)₂—, wherein the remaining 2 bonds are be independently substituted.

“Hydrocarbyloxy” (alone or in combination) means a hydrocarbyl ether group. Examples of suitable hydrocarbyl ether groups include methoxy, ethoxy, n-propoxy, isopropoxy, allyloxy, n-butoxy, iso-butoxy, sec-butoxy, tert-butoxy, and the like.

“Cyclohydrocarbyl” (alone or in combination) means a hydrocarbyl group that contains 3 to about 8 carbon atoms (preferably from about 3 to about 6 carbon atoms), and is cyclic. Examples of such cyclohydrocarbyl groups include cyclopropyl, cyclobutyl, cyclopentenyl, cyclohexyl, cyclooctynyl, and the like.

“Cyclohydrocarbylhydrocarbyl” means a hydrocarbyl group that is substituted by a cyclohydrocarbyl.

“Aryl” (alone or in combination) means a phenyl or naphthyl group that optionally contains one or more substituents selected from hydrocarbyl, hydrocarbyloxy, halogen, hydroxy, amino, nitro, and the like, such as phenyl, p-tolyl, 4-methoxyphenyl, 4-(tert-butoxy)phenyl, 4-fluorophenyl, 4-chlorophenyl, 4-hydroxyphenyl, and the like.

“Arylhydrocarbyl” (alone or in combination) means a hydrocarbyl in which one hydrogen atom is replaced by an aryl group, such as benzyl, 2-phenylethyl, and the like.

“Arylhydrocarbyloxycarbonyl” (alone or in combination) means —C(O)—O—arylhydrocarbyl. An example of an arylhydrocarbyloxycarbonyl is benzyloxycarbolyl.

“Aryloxy” means aryl-O—.

“Aromatic ring” means aryl or heteroaryl.

“Hydrocarbyloyl” or “hydrocarbylcarbonyl” (alone or in combination) means an acyl group derived from an hydrocarbylcarboxylic acid, examples of which include acetyl, propionyl, acryloyl, butyryl, valeryl, 4-methylvaleryl, and the like.

“Cyclohydrocarbylcarbonyl” means an acyl group derived from a (a) monocyclic or bridged cyclohydrocarbylcarboxylic acid such as cyclopropanecarbonyl, cyclohexenecarbonyl, adamantanecarbonyl, and the like; or (b) benz-fused monocyclic cyclohydrocarbylcarboxylic acid that is optionally substituted by, for example, a hydrocarbyloylamino group, such as 1,2,3,4-tetrahydro-2-naphthoyl, 2-acetamido-1,2,3,4-tetrahydro-2-naphthoyl.

“Arylhydrocarbyloyl” or “arylhydrocarbylcarbonyl” means an acyl group derived from an aryl-substituted hydrocarbylcarboxylic acid such as phenylacetyl, 3-phenylpropenyl (cinnamoyl), 4-phenylbutyryl, (2-naphthyl)acetyl, 4-chlorohydrocinnamoyl, 4-aminocinnamoyl, 4-methoxycinnamoyl, and the like.

“Aroyl” or “arylcarbonyl” means an acyl group derived from an aromatic carboxylic acid. Examples of such groups include aromatic carboxylic acids, an optionally substituted benzoic or naphthoic acid such as benzoyl, 4-chlorobenzoyl, 4-carboxybenzoyl, 4-(benzyloxycarbonyl)benzoyl, 2-naphthoyl, 6-carboxy-2 naphthoyl, 6-(benzyloxycarbonyl)-2-naphthoyl, 3-benzyloxy-2-naphthoyl, 3-hydroxy-2-naphthoyl, 3-(benzyloxyformamido)-2-naphthoyl, and the like.

The heterocyclyl (heterocyclo) or heterocyclohydrocarbyl portion of a heterocyclylcarbonyl, heterocyclyloxycarbonyl, heterocyclylhydrocarbyloxycarbonyl, heterocyclohydrocarbyl, the like is a saturated or partially unsaturated monocyclic, bicyclic, or tricyclic heterocycle that contains 1-4 hetero atoms (i.e., N, O, and/or S) and is optionally substituted (a) on one or more carbon atoms by a halogen, alkyl, alkoxy, oxo, an,d the like; (b) on a secondary nitrogen atom (i.e., —NH—) by a hydrocarbyl, arylhydrocarbyloxycarbonyl, hydrocarbyloyl, aryl, or arylhydrocarbyl; and/or (c) on a tertiary nitrogen atom (i.e. ═N—) by oxido and that is attached via a carbon atom. The tertiary nitrogen atom with 3 substituents also can form an N-oxide group (═N(O)—). Examples of such heterocyclyl groups include pyrrolidinyl, piperidinyl, piperazinyl, morpholinyl, thiamorpholinyl, and the like.

“Heteroaryl” means an aromatic heterocyclic ring that contains 1-4 atoms (i e., N, S, and/or O) in the ring that are other than carbon. A heteroaryl group can contain a single 5- or 6-membered ring or a fused ring system that contains two 6-membered rings or a 5- and a 6-membered ring. Exemplary heteroaryl groups include 6-membered ring substituents such as pyridyl, pyrazyl, pyrimidinyl, and pyridazinyl; 5-membered ring substituents such as 1,3,5-, 1,2,4- or 1,2,3-triazinyl, imidazyl, furanyl, thiophenyi, pyrazolyl, oxazolyl, isoxazolyl, thiazolyl, 1,2,3-, 1,2,4-, 1,2,5-, or 1,3,4-oxadiazolyl and isothiazolyl; 6/5-membered fused ring substituents such as benzothiofuranyl, isobenzothiofuranyl, benzisoxazolyl, benzoxazolyl, purinyl, and anthranilyl; and 6/6-membered fused rings such as 1,2-, 1,4-, 2,3- and 2,1-benzopyronyl, quinolinyl, isoquinolinyl, cinnolinyl, quinazolinyl, and 1,4-benzoxazinyl.

The heteroaryl portion of a heteroaroyl, heteroaryloxycarbonyl, heteroarylhydrocarbyloyl (heteroarylhydrocarbyl carbonyl), or the like is an aromatic monocyclic, bicyclic, or tricyclic heterocycle that contains the hetero atoms, and is optionally substituted as defined above with respect to the definition of heterocyclyl.

“Cyclohydrocarbylhydrocarbyloxy-carbonyl” means an acyl group derived from a cyclohydrocarbylhydrocarbyloxycarboxylic acid of the formula cyclohydrocarbylhydrocarbyl-O—COOH.

“Aryloxyhydrocarbyloyl” means aryl-O-hydrocarbyloyl.

“Heterocyclyloxycarbonyl” means an acyl group derived from heterocyclyl-O—COOH.

“Heterocyclyloxyhydrocaroyloy” means an acyl group derived from a heterocyclyl-substituted hydrocarbylcarboxylic acid.

“Heterocyclylhydrocarbyloxycarbonyl” means an acyl group derived from a heterocyclyl-substituted hydrocarbyl-O—COOH.

“Heteroaryloxycarbonyl” means an acyl group derived from a carboxylic acid represented by heteroaryl-O—COOH.

“Aminocarbonyl” (alone or in combination) means an amino-substituted carbonyl (carbamoyl) group derived from an amino-substituted carboxylic acid, wherein the amino group can be a primary, secondary, or tertiary amino group containing substituents selected from hydrogen, hydrocarbyl, aryl, aralkyl, cyclohydrocarbyl, cyclohydrocarbylhydrocarbyl groups, and the like.

“Aminohydrocarbyloyl” means an acyl group derived from an amino-substituted hydrocarbylcarboxylic acid, wherein the amino group can be a primary, secondary, or tertiary amino group containing substituents independently selected from hydrogen, alkyl, aryl, aralkyl, cyclohydrocarbyl, cyclohydrocarbylhydrocarbyl, and the like.

“Halogen” means fluorine, chlorine, bromine, or iodine.

“Halohydrocarbyl” means a hydrocarbyl group wherein one or more hydrogens are replaced with a halogen. Examples of such halohydrocarbyl groups include chloromethyl, 1-bromoethyl, fluoromethyl, difluoromethyl, trifluoromethyl, 1,1,1-trifluoroethyl, and the like. “Perfluorohydrocarbyl” means a hydrocarbyl group wherein each hydrogen has been replaced by a fluorine atom. Examples of such perfluorohydrocarbyl groups include trifluoromethyl, perfluorobutyl, perfluoroisopropyl, perfluorododecyl, and perfluorodecyl.

When naming the hydroxamates of Formula IV where R¹ comprises a cyclohydrocarbyl, heterocyclo, aryl, or heteroaryl which is directly bonded to the depicted SO₂, the positions on the ring are numbered such that the 1-position being the position that is bonded to the depicted SO₂.

### EXAMPLES

The following preferred specific examples are nearly illustrative, and not limiting to the remainder of this disclosure in any way.

### Example 1

### Racemic Mixture of N-hydroxy-2-[[(4-phenoxyphenyl)sulfonyl]-methyl]-1-(2-propynyl)-2-pyrrolidine carboxamide, monohydrochloride

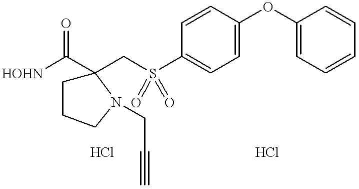

Part A: Lithium diisopropylamine (1.8M in THF (4.5 mL, 8.1 mmol)) was added to a solution of CBZ-proline methyl ester (2.0 g, 7.6 mmol) in THF (10 mL) at −76° C. The solution was then stirred for 1 hr. Next, diiodomethane (0.67 mL, 8.3 mmol) was added, and the solution was stirred for 20 hr at ambient temperature. The solution was subsequently concentrated, and the residue was dissolved into ethyl acetate, washed with H₂O, and dried over MgSO₄. Chromatography (ethyl acetate/hexane) provided the iodo compound as a yellow oil (980 mg, 32%).

Part B: NaH (60% suspension in mineral oil, 400 mg, 10 mmol) was added to a solution of 4-(phenoxy)benzenethiol (2.0 g, 9.9 mmol) in DMF (3 mL). The solution was then stirred at 0° C. for 30 min. The solution was subsequently added to a solution of the iodo compound of part A (4.0 g, 9.9 mmol) in DMF (4 mL), and the mixture was stirred for 18 hr at ambient temperature. The solution was then concentrated in vacuo, and the residue was dissolved into ethyl acetate, washed with H₂O, and dried over MgSO₄. Chromatography (ethyl acetate/hexane) provided the sulfide as a yellow oil (1.9 g, 40%

Part C: Oxone® was added to a solution of the sulfide of part B (1.9 g, 4.0 mmol) in methanol (300 mL) and H₂O (30 mL). The mixture was then stirred for 20 hr at ambient temperature. The excess solids were collected by filtration, and the filtrate was concentrated in vacuo. The residue was dissolved in ethyl acetate, washed with H₂O, and dried over MgSO₄. Concentration in vacuo provided the sulfone as a yellow solid (2.0 g, 98%).

Part D: The sulfone of part C (2.0 g, 3.9 mmol) was added to a solution of 10% Pd on C (410 mg, 0.38 mmol) in methanol (40 mL). The solution was then stirred under a H₂ atmosphere for 20 hr at ambient temperature. The resulting mixture was filtered, and the filtrate was concentrated. Chromatography (ethyl acetate/hexane) provided the amine as an oil (1.0 g, 69%).

Part E: Propargyl bromide (0.40 mL, 5.3 mmol) and K₂CO₃ (1.1 g, 7.9 mmol) was added to a solution of the amine of part D (1.0 g, 2.6 mmol) in DMF (10 mL). The solution was then stirred for 20 hr at ambient temperature. Next, the solution was concentrated in vacuo, and the residue was dissolved into 1M KHSO₄. The resulting solution was extracted with ethyl ether, and the aqueous layer was made basic with saturated NaHCO₃. The aqueous layer was extracted with ethyl acetate, and concentration in vacuo provided the propargyl amine as a solid (600 mg, 51%).

Part F: NaOH (440 mg, 11 mmol) in H₂O (10 mL) was added to a solution of the propargyl amine of part E (500 mg, 1.1 mmol) in methanol (5 mL) and THF (5 mL). The solution was then heated at reflux for 20 hr. Afterward, the solution was concentrated in vacuo, and the residue was dissolved into H2O. The solution was extracted with ethyl ether, and the aqueous portion was acidified with concentrated HCl to pH=3. The resulting white precipitate was collected by filtration to provide the acid (400 mg, 76%).

Part G: 1-(3-dimethylaminopropyl)-3-ethylcarbodiimide hydrochloride (510 mg, 2.7 mmol) and N-hydroxybenzotriazole (370 mg, 2.7 mmol) was added to a solution of the acid of part F (320 mg, 0.80 mmol) in DMF (10 mL). Afterward, N-methylmorpholine (0.36 mL, 3.2 mmol) and 50% aqueous hydroxylamine (0.5 mL) was added. The resulting solution was then stirred for 20 hr at ambient temperature. The solution was subsequently concentrated in vacuo, and the residue was dissolved in ethyl acetate, washed with H₂O, and dried over MgSO₄. After concentration in vacuo, the residue was dissolved in acetonitrile, and concentrated HCl was added to form the HCl salt. Reverse phase chromatography (acetonitrile/H₂O) provided a racemic mixture of the title compound as a white solid (130 mg, 36%). MS(CI) MH⁺ calcd. for C₂₁H₂₂N₂O₅S: 415, found 415. Anal. calc. for C₂₁H₂₂N₂O₅S.HCL: C, 55.93; H, 5.14; N, 6.21. Found: C, 55.76; H, 5.37; N, 5.72.

### Example 2

### Separation of the Monohydrochloride Salt Enantiomers of Example 1 to Form Separate Trifluoroacetate Salts

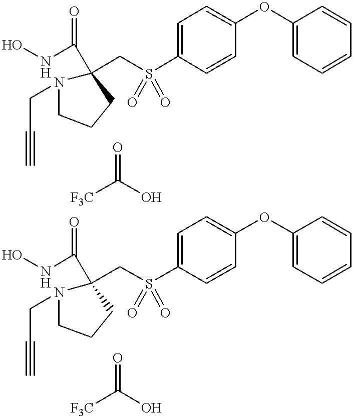

A sample the product of Example 1 was chromatographed on a chiral column using a Chiralpak AD column (manufacturer: Daicel, Japan: packing: silica gel particles coated with amylose tris(3,5-dimethylphenyl carbanate); particle size: 10μ; column inside diameter: 22.5 mm; column length: 250 mm) with a mobile phase of 35% ethanol and 65% Heptane with 0.2% trifluoroacetate (“TFA”) under isocratic conditions. The separation was monitored by chiral HPLC using a Chiralpak AD column (packing: silica gel particles coated with amylose tris(3,5-dimethylphenyl carbanate); particle size: 10 μm; column inside diameter: 4.6 mm; column length: 250 mm) with a mobile phase of 35% ethanol and 65% Heptane with 0.2% TFA. The first enantiomer (“Enantiomer 1”) to exit the column had a retention time of 9.59 min, and the second enantiomer (“Enantiomer 2”) to exit the column had a retention time of 12.37 min under these conditions. Enatiomer 1 had a mass spectrometry result (calculated for C₂₁H₂₂N₂O₅S: 414) of m/e 414, and Enatiomer 2 had a mass spectrometry result (calculated for C₂₁H₂₂N₂O₅S: 414) of m/e 414.

### Example 3

### In Vitro MMP Inhibition

The salts prepared in the manner described in Examples 1 and 2 were assayed for activity by an in vitro assay following the procedures of Knight et al., FEBS Lett. 296(3):263 (1992). Briefly, 4-aminophenylmercuric acetate (APMA) or trypsin activated MMPs were incubated with various concentrations of the inhibitor at room temperature for 5 min.

Recombinant human MMP-13 and MMP-1 enzymes were prepared in laboratories of the assignee. MMP-13 was expressed in baculovirus as a proenzyme, and purified first over a heparin agarose column and then over a chelating zinc chloride column. The proenzyme was activated by APMA for use in the assay. MMP-1 expressed in transfected HT-1080 cells was provided by Dr. Howard Welgus of Washington University, St. Louis, Mo. The enzyme was also activated using APMA, and then purified over a hydroxamic acid column.

The enzyme substrate is a methoxycoumarin-containing polypeptide having the following sequence:

MCA-ProLeuGlyLeuDpaAlaArgNH₂, wherein MCA is methoxycoumarin and Dpa is 3-(2,4-dinitrophenyl)-L-2,3-diaminopropionyl alanine. This substrate is commercially available from Baychem as product M-1895.

The buffer used for assays contained 100 mM Tris-HCl, 100 mM NaCl, 10 mM CaCl₂ and 0.05% polyethyleneglycol (23) lauryl ether at a pH value of 7.5. Assays were carried out at room temperature, and dimethyl sulfoxide (DMSO) at a final concentration of 1% was used to dissolve inhibitor.

The assayed inhibitor in DMSO/buffer solution was compared to an equal amount of DMSO/buffer with no inhibitor as control using Microfluor™ White Plates (Dynatech). The inhibitor or control solution was maintained in the plate for 10 min and the substrate was added to provide a final concentration of 4 microM.

In the absence of inhibitor activity, a fluorogenic peptide was cleaved at the gly-leu peptide bond, separating the highly fluorogenic peptide from a 2,4-dinitrophenyl quencher, resulting in an increase of fluorescence intensity (excitation at 328 nm/emission at 415 nm). Inhibition was measured as a reduction in fluorescent intensity as a function of inhibitor concentration, using a Perkin Elmer L550 plate reader. The IC₅₀ values were calculated from those values. The results are set forth in the Table 1 below, reported in terms of IC₅₀ values in nanomolar (nm) amounts.

TABLE 1

| Example | MMP-1 | MMP-2 | MMP-13 |
|---|---|---|---|
| 1 | 400 | 0.2 | 1.3 |
| 2 (Enantiomer 1) | 300 | <0.1 | <0.1 |
| 2 (Enantiomer 2) | >10,000 | 19.3 | 60.0 |

* * * * * * * *

The above detailed description of preferred embodiments is intended only to acquaint others skilled in the art with the invention, its principles, and its practical application so that others skilled in the art may adapt and apply the invention in its numerous forms, as they may be best suited to the requirements of a particular use. This invention, therefore, is not limited to the above embodiments, and may be variously modified.

With reference to the use of the word(s) “comprise” or “comprises” or “comprising” in the above description and/or in the following claims, Applicants note that unless the context requires otherwise, those words are used on the basis and clear understanding that they are to be interpreted inclusively, rather than exclusively, and that Applicants intend each of those words to be so interpreted in construing the above description and/or the following claims.

We claim:

1. A compound or salt thereof, the compound having a structure selected from the group consisting of:

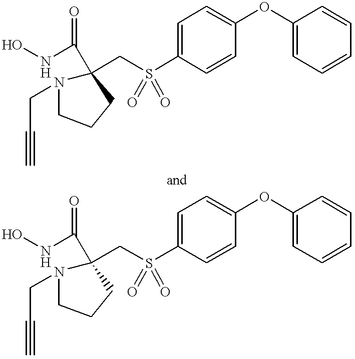

2. A salt according to claim 1, wherein the salt is a trifluoroacetate salt selected from the group consisting of:

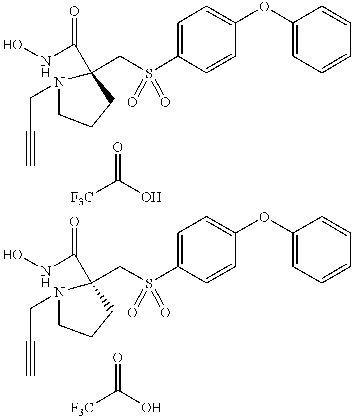

3. A salt according to claim 1, wherein the salt is a monohydrochloride salt selected from the group consisting of:

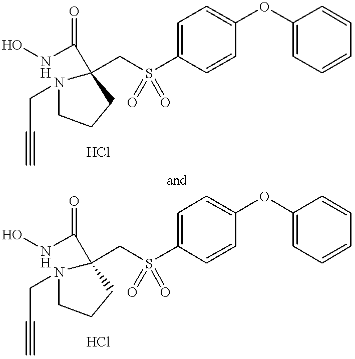

4. A compound or salt thereof according to claim 1, wherein the compound is identifiable in that a trifluoroacetate salt of the compound exits from a chiral high performance liquid chromatography column before a trifluoroacetate salt of the other compound recited in claim 1 under the following conditions:
(a) a mixture of monohydrochloride salts of both compounds is introduced with an isocratic eluent into the column;
(b) 34.8 to 35% (by weight) of the eluent consists of ethanol, 64.8 to 65% (by weight) of the eluent consists of heptane, and 0.2% (by weight) of the eluent consists of trifluoroacetic acid;
(c) the column has a 250 mm length and a 22.5 mm inside diameter; and
(d) the column is packed with silica gel particles, which have a 10 micron particle size and are coated with amylose tris(3,5-dimethylphenyl carbamate).

5. A salt according to claim 4, wherein the salt comprises a trifluoroacetate salt.

6. A compound or salt thereof according to claim 1, wherein the compound is identifiable in that a trifluoroacetate salt of the compound exits from a chiral high performance liquid chromatography column after a trifluoroacetate salt of the other compound recited in claim 1 under the following conditions:
(a) a mixture of monohydrochloride salts of both compounds is introduced with an isocratic eluent into the column;
(b) 34.8 to 35% (by weight) of the eluent consists of ethanol, 64.8 to 65% (by weight) of the eluent consists of heptane, and 0.2% (by weight) of the eluent consists of trifluoroacetic acid;
(c) the column has a 250 mm length and a 22.5 mm inside diameter; and
(d) the column is packed with silica gel particles, which have a 10 micron particle size and are coated with amylose tris(3,5-dimethylphenyl carbamate).

7. A salt according to claim 6, wherein the salt comprises a trifluoroacetate salt.

8. A pharmaceutical composition comprising a compound or pharmaceutically acceptable salt thereof, wherein the compound has a structure selected from the group consisting of:

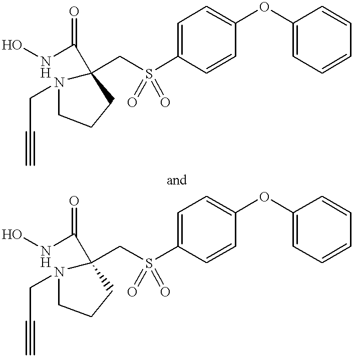

9. A pharmaceutical composition according to claim 8, wherein the compound is identifiable in that a trifluoroacetate salt of the compound exits from a chiral high performance liquid chromatography column before a trifluoroacetate salt of the other compound recited in claim 8 under the following conditions:
(a) a mixture of monohydrochloride salts of both compounds is introduced with an isocratic eluent into the column;
(b) 34.8 to 35% (by weight) of the eluent consists of ethanol, 64.8 to 65% (by weight) of the eluent consists of heptane, and 0.2% (by weight) of the eluent consists of trifluoroacetic acid;
(c) the column has a 250 mm length and a 22.5 mm inside diameter; and
(d) the column is packed with silica gel particles, which have a 10 micron particle size and are coated with amylose tris(3,5-dimethylphenyl carbamate).

10. A pharmaceutical composition according to claim 8, wherein the compound is identifiable in that a trifluoroacetate salt of the compound exits from a chiral high performance liquid chromatography column after a trifluoroacetate salt of the other compound recited in claim 8 under the following conditions:
(a) a mixture of monohydrochloride salts of both compounds is introduced with an isocratic eluent into the column;
(b) 34.8 to 35% (by weight) of the eluent consists of ethanol, 64.8 to 65% (by weight) of the eluent consists of heptane, and 0.2% (by weight) of the eluent consists of trifluoroacetic acid;
(c) the column has a 250 mm length and a 22.5 mm inside diameter; and
(d) the column is packed with silica gel particles, which have a 10 micron particle size and are coated with amylose tri(3,5-dimethylphenyl carbamate).

11. A method for treating or preventing a pathological condition associated with matrix metalloproteinase activity in a mammal, the method comprising administering a matrix metalloproteinase inhibitor compound or a pharmaceutically acceptable salt thereof in an effective amount to the mammal, wherein:
the compound has a structure selected from the group consisting of:

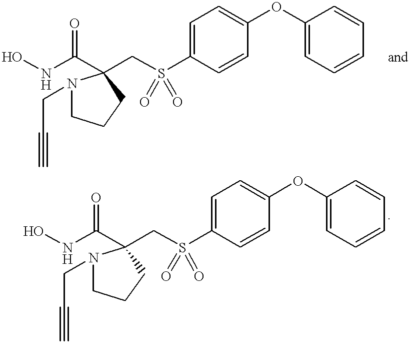

12. A method according to claim 11, wherein
the mammal does not have a pathological condition associated with matrix metalloproteinase activity, but is disposed to having a pathological condition associated with matrix metalloproteinase activity; and
the method comprises administering the matrix metalloproteinase inhibitor compound or salt thereof in an effective amount to prevent the pathological condition that the mammal is disposed to having.

13. A method according to claim 11, wherein
the mammal has a pathological condition associated with matrix metalloproteinase activity; and
the method comprises administering the matrix metalloproteinase inhibitor compound or salt thereof in an effective amount to treat the pathological condition.

14. A method according to claim 11, wherein the compound is identifiable in that a trifluoroacetate salt of the compound exits from a chiral high performance liquid chromatography column before a trifluoroacetate salt of the other compound recited in claim 11 under the following conditions:
(a) a mixture of monohydrochloride salts of both compounds is introduced with an isocratic eluent into the column;
(b) 34.8 to 35% (by weight) of the eluent consists of ethanol, 64.8 to 65% (by weight) of the eluent consists of heptane, and 0.2% (by weight) of the eluent consists of trifluoroacetic acid;
(c) the column has a 250 mm length and a 22.5 mm inside diameter; and
(d) the column is packed with silica gel particles, which have a 10 micron particle size and are coated with amylose tris(3,5-dimethylphenyl carbamate).

15. A method according to claim 11, wherein the compound is identifiable in that a trifluoroacetate salt of the compound exits from a chiral high performance liquid chromatography column after a trifluoroacetate salt of the other compound recited in claim 11 under the following conditions:
(a) a mixture of monohydrochloride salts of both compounds is introduced with an isocratic eluent into the column;
(b) 34.8 to 35% (by weight) of the eluent consists of ethanol, 64.8 to 65% (by weight) of the eluent consists of heptane, and 0.2% (by weight) of the eluent consists of trifluoroacetic acid;
(c) the column has a 250 mm length and a 22.5 mm inside diameter; and
(d) the column is packed with silica gel particles, which have a 10 micron particle size and are coated with amylose tris(3,5-dimethylphenyl carbamate).

16. A method for inhibiting matrix metalloproteinase 2 activity selectively over matrix metalloproteinase 1 activity in a mammal, the method comprising administering a matrix metalloproteinase inhibitor compound or pharmaceutically acceptable salt thereof to the mammal, wherein:
the compound has a structure selected from the group consisting of:

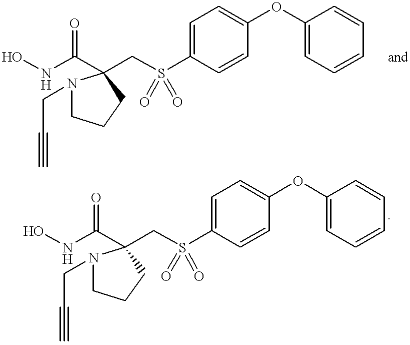

17. A method according to claim 16, the process further comprising inhibiting matrix metalloproteinase 13 activity selectively over matrix metalloproteinase 1 activity in the mammal by said administration of the matrix metalloproteinase inhibitor compound or salt thereof.

18. A method according to claim 16, wherein the compound is identifiable in that a trifluoroacetate salt of the compound exits from a chiral high performance liquid chromatography column before a trifluoroacetate salt of the other compound recited in claim 16 under the following conditions:
(a) a mixture of monohydrochloride salts of both compounds is introduced with an isocratic eluent into the column;
(b) 34.8 to 35% (by weight) of the eluent consists of ethanol, 64.8 to 65% (by weight) of the eluent consists of heptane, and 0.2% (by weight) of the eluent consists of trifluoroacetic acid;
(c) the column has a 250 mm length and a 22.5 mm inside diameter; and
(d) the column is packed with silica gel particles, which have a 10 micron particle size and are coated with amylose tris(3,5-dimethylphenyl carbamate).

19. A method according to claim 18, the process further comprising inhibiting matrix metalloproteinase 13 activity selectively over matrix metalloproteinase 1 activity in the mammal by said administration of the matrix metalloproteinase inhibitor compound or salt thereof.

20. A method according to claim 16, wherein the compound is identifiable in that a trifluoroacetate salt of the compound exits from a chiral high performance liquid chromatography column after a trifluoroacetate salt of the other compound recited in claim 16 under the following conditions:
(a) a mixture of monohydrochloride salts of both compounds is introduced with an isocratic eluent into the column;
(b) 34.8 to 35% (by weight) of the eluent consists of ethanol, 64.8 to 65% (by weight) of the eluent consists of heptane, and 0.2% (by weight) of the eluent consists of trifluoroacetic acid;
(c) the column has a 250 mm length and a 22.5 mm inside diameter; and
(d) the column is packed with silica gel particles, which have a 10 micron particle size and are coated with amylose tris(3,5-dimethylphenyl carbamate).

21. A method according to claim 20, the process further comprising inhibiting matrix metalloproteinase 13 activity selectively over matrix metalloproteinase 1 activity in the mammal by said administration of the matrix metalloproteinase inhibitor compound or salt thereof.

22. A method for inhibiting matrix metalloproteinase 13 activity selectively over matrix metalloproteinase 1 activity in a mammal, the method comprising administering a matrix metalloproteinase inhibitor compound or a pharmaceutically acceptable salt thereof in an effective amount to the mammal, wherein:
the compound has a structure selected from the group consisting of:

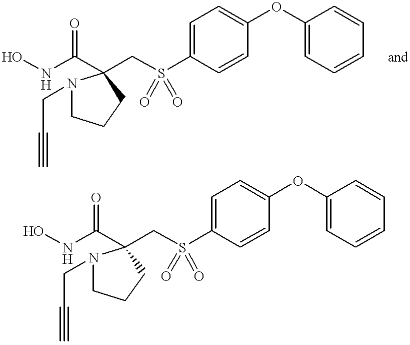

23. A method according to claim 22, wherein the compound is identifiable in that a trifluoroacetate salt of the compound exits from a chiral high performance liquid chromatography column before a trifluoroacetate salt of the other compound recited in claim 22 under the following conditions:
(a) a mixture of monohydrochloride salts of both compounds is introduced with an isocratic eluent into the column;
(b) 34.8 to 35% (by weight) of the eluent consists of ethanol, 64.8 to 65% (by weight) of the eluent consists of heptane, and 0.2% (by weight) of the eluent consists of trifluoroacetic acid;
(c) the column has a 250 mm length and a 22.5 mm inside diameter; and
(d) the column is packed with silica gel particles, which have a 10 micron particle size and are coated with amylose tris(3,5-dimethylphenyl carbamate).

24. A method according to claim 22, wherein the compound is identifiable in that a trifluoroacetate salt of the compound exits from a chiral high performance liquid chromatography column after a trifluoroacetate salt of the other compound recited in claim 22 under the following conditions:
(a) a mixture of monohydrochloride salts of both compounds is introduced with an isocratic eluent into the column;
(b) 34.8 to 35% (by weight) of the eluent consists of ethanol, 64.8 to 65% (by weight) of the eluent consists of heptane, and 0.2% (by weight) of the eluent consists of trifluoroacetic acid;
(c) the column has a 250 mm length and a 22.5 mm inside diameter; and
(d) the column is packed with silica gel particles, which have a 10 micron particle size and are coated with amylose tris(3,5-dimethylphenyl carbamate).
